(19) United States
(12) Patent Application Publication
(10) Pub. No.: US 2022/0151695 A1
(43) Pub. Date: May 19, 2022

(54) ENERGY DELIVERY SYSTEM AND METHOD

(71) Applicant: Emblation Limited, Alloa (GB)

(72) Inventors: Gary Beale, Alloa (GB); Eamon McErlean, Alloa (GB)

(73) Assignee: Emblation Limited, Alloa (GB)

(21) Appl. No.: 17/439,563

(22) PCT Filed: Mar. 11, 2020
(86) PCT No.: PCT/GB2020/050610

§ 371 Date: Sep. 15, 2021

(30) Foreign Application Priority Data
Mar. 15, 2019 (GB) 1903540.1

(51) Int. Cl. A61B 18/18; A61B 34/30; A61B 18/12

(52) U.S. Cl. CPC A61B 18/1815; A61B 34/30; A61B 2018/00095; A61B 2018/1823; A61B 18/1206

(57) ABSTRACT

A system comprises a surgical robot comprising a moveable robotic arm; a radiating applicator positioned at a distal end of the robotic arm, wherein the robotic arm is configured to move the radiating applicator to a desired operational position; and an energy source positioned on a distal portion of the robotic arm, in proximity to the radiating applicator, wherein the energy source is configured to provide RF or microwave energy to the radiating applicator for radiation by the radiating applicator.

### FIELD

This invention relates to energy delivery via a surgical robotic system to an antenna, for example an interstitial or catheter antenna, from a medical electromagnetic energy system to deliver energy into biological tissues for ablative or non-ablative purposes.

### BACKGROUND

It is known to provide microwave ablation system in which microwave energy is used to perform ablation or heating of tissue.

In most energy ablation systems the energy is delivered from an energy generator, via a connecting coaxial cable, to a radiating applicator that transfers the energy into the tissue. In these applicators, the radiating element may be surrounded by tissue, pierce tissue or be placed in contact with the tissue. For such systems, a typical standard practice is to deliver energy for a treatment lasting typically anywhere from 1 to 20 minutes to raise the temperature of tissue greater than 43° C. to 45° C., for example to 60° C., 70° C. or 100° C. and beyond such that necrosis occurs within the desired ablation zone. The energy may be delivered to have an amplitude-modulated or pulse width-modulated duty cycle such that a required level of energy is maintained or controlled for the duration of the energy release.

One undesired aspect of high frequency electromagnetic energy delivery in coaxial cabling is that energy is lost within the cabling via heat along its length. The interconnecting coaxial cabling that connects the energy generator to the radiating applicator may form part of the treatment applicator or may be a lower loss reusable cable that connects to the higher loss (smaller) treatment applicator. Another disadvantage is high frequency coaxial cabling can be easily damaged by being crushed or kinked which can cause reflection or absorption of energy.

In surgeries that involve robotic placement systems, these mechanisms can complicate the delivery of energy to the treatment applicator as the dynamics and practicalities of the robotic freedom of movement must be considered.

Two primary options are typically available. The first is to place the energy generator system near to the treatment location and then feed the cabling to the end-effector, such an arrangement is described in JP2009178506A. The disadvantage of this is that it may limit the range of articulation of the robotic limbs and take the generator and cabling close to the robots' sterile field. This may also add complexity in placing or moving the patient and attaching or disconnecting the energy delivery cabling during the treatment. It also risks damaging the cabling or the generator should the robot manipulator be parked at a location a distance away from the treatment site without the cabling being disconnected first.

An example of a surgical robot 1 is illustrated in FIG. 1. In this example an electromagnetic energy system 2 is connected via a cabling means 3 to the robot 1. The electromagnetic energy system 2 may also be referred to as an electromagnetic source 2. The end effector of the robot and the energy applicator are within the sterile field 4 of treatment which constrains location of the non-sterile components or devices involved in the treatment. The energy 5 generated by a standard electromagnetic source 2 is typically of a greater level, for example. 180 W, than the energy 6 delivered to the treatment site, which may be for example 100 W. The energy generated by the electromagnetic energy system 2 may be significantly higher than the energy delivered to the applicator, to accommodate the energy attenuated via the cabling path loss.

The second option is to place the energy generator system within the main body of the robot 1, or mounted on to or adjacent to the main body. In this arrangement the energy cabling may be fed inside the joints or along the outside of the robot via the limbs to the end effector at the treatment site. This again may limit the freedom of the robot and may require a long cable path to traverse the entire length of the robot from the energy generator. In both cases additional energy may have to be created by the generator to accommodate the overall path losses to ensure sufficient energy is delivered to the treatment site. In RF and microwave systems this increased energy requirement may add complexity and expense to the system in addition to size due to additional cooling and heat sink requirements. The risk of damaging such a long cable also exists which could impact the treatment efficacy or reliability by completely preventing the treatment occurring.

In an alternative example shown in FIG. 2 a standard electromagnetic energy source 7 is placed into or located onto a surgical robot. In this scenario the cabling 8 runs along the length of the robotic system 1 to the end effector at the treatment location which may incur the same or greater path loss as FIG. 1.

### SUMMARY OF THE INVENTION

In a first aspect, there is provided a system comprising: a surgical robot comprising a moveable robotic arm; a radiating applicator positioned at a distal end of the robotic arm, wherein the robotic arm is configured to move the radiating applicator to a desired operational position; and an energy source positioned on a distal portion of the robotic arm, in proximity to the radiating applicator; wherein the energy source is configured to provide RF or microwave energy to the radiating applicator for radiation by the radiating applicator.

The system may further comprise a coaxial cable connecting the energy source to the radiating applicator. A length of the coaxial cable may be less than 2 m, optionally less than 1 m, further optionally less than 0.5 m.

The energy source may be connected directly to the radiating applicator.

The distal portion of the robotic arm may comprise an end-effector of the robot arm. The radiating applicator may be positioned on the end-effector. The energy source may be positioned on the end-effector.

The distal portion of the robotic arm may further comprise a further link of the robotic arm.

The energy source may be positioned on the further link. The further link may be adjacent to the end-effector.

The distal portion of the robotic arm may be axially rotatable with respect to a preceding portion of the robotic arm.

The end-effector may be axially rotatable with respect to the further link of the robotic arm, so as to provide rotation of the radiating applicator independently of rotation of the energy source.

The system may further comprise a rotatable coaxial coupling between the coaxial cable and the energy source. The system may further comprise a rotatable coaxial coupling between the coaxial cable and the radiating applicator.

The radiating applicator may comprise a directional antenna. The robotic arm may be configured to rotate the radiating applicator to provide a desired direction of radiation from the directional antenna.

The system may further comprise a thermal interface between the energy source and robotic arm. The thermal interface may be configured to pass heat from the energy source into the robotic arm for the purpose of heat sinking. The thermal interface may comprise a high thermal conductivity material. The high thermal conductivity material may comprise at least one of a Cu-Cu bracket, metallised pyrolytic carbon, metallised thermally annealed pyrolytic graphite (APG).

The energy source may comprise an energy generator configured to receive electrical energy and to generate RF or microwave energy.

The energy source may comprise an amplifier configured to receive lower-power RF or microwave energy and to generate higher-power RF or microwave energy.

The robotic arm may comprises a power connection for powering peripheral devices and/or tools. The energy source may be configured to connect to the power connection. The power connection may comprise at least one of a battery, a port, an electrical bus.

The system may further comprise a controller configured to control at least one parameter of the energy provided by the energy source. The at least one parameter may comprise at least one of: power, frequency, duty cycle, gain control, antenna direction, antenna rotational speed, advancement rate, withdrawal rate. The controller may be configured to control the at least one parameter to provide a desired amount of heating, for example to perform a desired ablation.

The system may further comprise a communications device. The communications device may be configured to obtain data relating to the energy provided by the energy source. The communications device may be configured to obtain data relating to an effect of the energy provided by the energy source. The communications device may be configured to send the data to the controller. The controller may be configured to control the at least one parameter of the energy provided by the energy source based on the data sent by the communications device.

The robotic arm may comprise a data connection or network connection. The communications device may be configured to connect to the data connection or network connection. The communications device may be configured to send the data to the controller via the data connection or network connection.

The communications device may comprise or be coupled to at least one sensor. The at least one sensor may comprise at least one of a power sensor, a temperature sensor, a pressure sensor.

The data may comprise at least one of a system temperature, an applicator temperature, forward power, reflected power, duty cycle, antenna direction, antenna rotational speed, advancement rate, withdrawal rate.

The communications device may comprise or form part of the energy source.

The system may further comprise a position detector system configured to output position data that is representative of a position of the radiating applicator. The controller may be configured to control the energy source in dependence on the position data. Controlling the energy source in dependence on the position data may comprise controlling energy during applicator withdrawal to perform surgery tract ablation.

Controlling the energy source in dependence on the position data may comprise controlling energy delivered by the energy source based on volume data held in a planning system. The volume data may comprise the desired operational position. The volume data may comprise tumour volume data.

The robotic arm may comprise a mechanical mounting adapter configured for mounting at least one peripheral device and/or tool. The energy source may be mounted on the mechanical mounting adapter. The at least one peripheral device and/or tool may comprise at least one of a camera, an endoscope.

The system may further comprise a cooling system positioned on the distal portion of the robotic arm, in proximity to the radiating applicator. The cooling system may be configured to cool the radiating applicator by circulation of a coolant fluid through at least one coolant channel.

A length of the at least one coolant channel may be less than 3 m, optionally less than 2 m, further optionally less than 1 m, further optionally less than 0.5 m.

The distal portion of the robotic arm may further comprise a further link of the robotic arm. At least part of the cooling system may be positioned on the further link. The energy source may be positioned on the further link.

The end-effector may be axially rotatable with respect to the further link of the robotic arm, so as to provide rotation of the radiating applicator independently of rotation of the at least part of the cooling system.

The system may comprise a rotatable coupling configured to connect the at least one coolant channel to the at least part of the cooling system. The rotatable coupling may be further configured to connect the energy source to the coaxial cable. The rotatable coupling may be further configured to connect the radiating applicator to the coaxial cable.

In a further aspect of the invention, which may be provided independently, there is provided a system comprising: a surgical robot comprising a moveable robotic arm; a radiating applicator positioned at a distal end of the robotic arm, wherein the robotic arm is configured to move the radiating applicator to a desired operational position; and a cooling system positioned on a distal portion of the robotic arm, in proximity to the radiating applicator; wherein the cooling system is configured to cool the radiating applicator by circulation of a coolant fluid through at least one coolant channel.

A length of the coolant channel may be less than 3 m, optionally less than 2 m, further optionally less than 1 m, further optionally less than 0.5 m.

The distal portion of the robotic arm may comprise an end-effector of the robot arm. The radiating applicator may be positioned on the end-effector.

The distal portion of the robotic arm may further comprise a further link of the robotic arm. At least part of the cooling system may be positioned on the further link.

The end-effector may be axially rotatable with respect to the further link of the robotic arm, so as to provide rotation of the radiating applicator independently of rotation of the at least part of the cooling system.

The system may comprise a rotatable coupling configured to connect the at least one coolant channel to the at least part of the cooling system.

The at least part of the cooling system may comprise a pump and a coolant reservoir. The coolant channel may be coupled to the pump. The coolant channel may be coupled to the coolant reservoir.

The frequency may be between 900 MHz and 30 GHz. The frequency may be about 915 MHz. The frequency may be about 2.45 GHz. The frequency may be about 5.8 GHz. The frequency may be about 8.0 GHz. The frequency may be about 24.125 GHz.

A diameter of the coaxial cable may be between 0.1 mm and 15 mm.

The tissue heating may be so as to perform tissue ablation. The tissue heating may be so as to cause tissue hyperthermia.

A more compact energy source for the delivery of microwave energy into tissue may be provided. The energy source may be an energy generator or a compact power amplifier that creates sufficient energy as required for a treatment.

A more efficient approach described herein may be to advantageously place or integrate a compact energy source onto or close to the end effector of a surgical robotic system. The path from this to the treatment applicator could be significantly reduced which in turn can reduce the energy required to be created for delivery. These benefits would also in part be enabled by the use of a robot as this type of machine could mechanically hold the mass of the compact energy generator close to the patient for a prolonged period of time in a way a clinician might not be physically be able to.

The energy generator may be constructed from lightweight materials For example conductive metal substrates or housings may be fabricated from aluminium metal foam or gold or silver plated titanium. Constructing the energy generator from lightweight materials may permit the robotic manipulator to move without any significant inertial burden. The energy generator may be constructed to be compatible with a camera or endoscope mounting adapter or mounting location and may be designed to be of similar compatible mass to existing tools for compatibility.

The energy generator may also be able to take advantage of the construction of the robot to dissipate its excess heat into metallic or thermally conductive elements of the robot to further reduce size. The energy generator may have particular thermal interface points that could mate with similar corresponding points on the robot, e.g. Cu-Cu brackets or pyrolytic carbon or thermally annealed pyrolytic graphite (APG) materials or combinations thereof e.g. Cu-APG or Aluminium-APG interface plates. These anchor structures may advantageously exploit the larger thermal mass of the robot to sink heat away from the energy generator.

The energy generator may derive its power source from a battery or port or electrical power buses within the robot intended to power or communicate to peripherals or tools. It may also access communications or networks within the robot to communicate with an external controller (not shown) to provide feedback of treatment parameters such as system/applicator temperatures, forward and reflected power, duty cycle or other relevant parameters. Sensors such as diode detectors and couplers may be used to measure forward and reverse microwave power. Temperature sensors such as thermistors and thermocouple sensors may be used to relay temperature of cabling or circuitry. Mechanical pressure sensors may be used to determine contact or resistance to motion. The data from these sensors may be used to control the location of a probe within the body or to adjust the rate of travel along an axis of movement to ensure constant energy delivery or to prevent overheating or to determine the progress of an ablation. The sensors may be positioned within the energy generator. The sensors may be positioned outside the energy generator. The sensors may be positioned on the robotic arm. The sensors may be positioned on the applicator.

A communications device (not shown) may be used to communicate data from a sensor or sensors to the energy source and/or to the external controller.

Likewise the position of the energy delivery probe known by the robotic system may be fed back to the energy generator system to adaptively control the energy delivery (duty cycle or amplifier gain control), for example to control energy during applicator withdrawal for the purposes of surgery tract ablation or to control the energy delivered to a known three dimensional physiological location based upon tumour volume data held in a planning system.

In many ablation systems the applicators and cabling are cooled to enhance delivery of energy and to also ensure the patient surfaces are not inadvertently heated in unintended non-treatment locations. This may include for example external skin surfaces at percutaneous or laparoscopic access points, mucosal or respiratory tracts, bronchial passageways, intravenous or arterial access pathways. The robotic limbs may hold the cooling pump and cooling medium (fluid or gas) reservoirs in close proximity to the treatment applicator to reduce the cumbersome cabling required to supply the cooling medium to the antenna. There may be an advantage from the reduced energy requirements to reduce the level of cooling required or conversely the same level of cooling could further improve efficiencies.

The surgical robot may employ axial control of the radiation applicator in the end effector to control the location or direction of radiation of a directional applicator. The antenna and energy generator may be rotated about this axis or conversely the energy generator may be fixed in a axially static location on a limb that has fewer degrees of freedom and the antenna may be placed on a connected limb or end effector with an additional rotational axis. The applicator cabling may axially twist about this additional axis depending upon rotational range required or it may be designed with a rotating connection to ensure constant energy delivery across 360+ degrees of rotation, for example 720+ degrees of axial rotation.

This rotational consideration may also extend to the cooling medium channels which may rotate or twist with the antenna or may employ secondary rotating couplings for the cooling medium to ensure greater axial freedom.

In a further aspect of the invention, which may be provided independently, there is provided an electromagnetic energy delivery system placed on or near the end effector or terminal limb of a surgical robot.

In a further aspect of the invention, which may be provided independently, there is provided an electromagnetic energy delivery system including an antenna cooling apparatus placed on or near the end effector or terminal limb of a surgical robot.

The electromagnetic energy delivery system may impart thermal energy to the robot for the purpose of heat sinking.

The energy generator system may derive its power source from a battery or port or electrical power bus within the robot intended to power or communicate to peripherals or tools.

The energy generator system may also access communications or networks within the robot to communicate with an external controller to provide feedback of treatment parameters such as system/applicator temperatures, forward and reflected power, duty cycle or other relevant parameters.

The position of the energy delivery probe known by the robotic system may be fed back to the energy generator system to adaptively control the energy delivery via duty cycle or amplifier gain control. The position of the energy delivery probe may be used to control energy during applicator withdrawal for the purposes of surgery tract ablation.

The thermal interface points may mate with similar corresponding points on the robot using Cu-Cu brackets, pyrolytic carbon, thermally annealed pyrolytic graphite (APG) materials or combinations thereof such as Cu-APG or Aluminium-APG interface plates.

The electromagnetic energy delivery system may be constructed to be compatible with a camera or endoscope mounting adapter.

The electromagnetic energy delivery system may be placed on or near the end effector or terminal limb of a surgical robot wherein the energy delivery system rotates in unison with a directional antenna.

In a further aspect of the invention, which may be provided independently, there is provided an electromagnetic energy delivery system placed on or near the end effector or terminal limb of a surgical robot wherein the energy delivery system is fixed and the end effector or terminal limb rotates independently. The rotating end effector or terminal limb may be for use with a directional antenna. A rotating coaxial coupling may be used. The energy delivery system and cooling system may be fixed and the end effector or terminal limb may rotate independently. Rotational coupling may be employed on the cooling medium channels. The rotational coupling for the cooling medium channels may be a secondary rotating coupling for the cooling medium or may be integrated with the primary rotating coupling for the electromagnetic energy delivery.

There may be provided a method or system substantially as described herein with reference to the accompanying drawings.

Features in one aspect may be provided as features in any other aspect as appropriate. For example, features of a method may be provided as features of an apparatus and vice versa. Any feature or features in one aspect may be provided in combination with any suitable feature or features in any other aspect.

### BRIEF DESCRIPTION OF DRAWINGS

Embodiments of the invention are now described, by way of non-limiting example, and are illustrated in the following figures, in which:—

FIG. 1 is a diagrammatic illustration of an electromagnetic energy generator used with a surgical robot to place an applicator;

FIG. 2 is a diagrammatic illustration of a variation of an electromagnetic energy generator used with a surgical robot to place an applicator;

FIG. 3 is a diagrammatic illustration of an energy generator located on a robotic limb in accordance with an embodiment;

FIG. 4 is a diagrammatic illustration of an energy generator and cooling system located on a robotic limb in accordance with an embodiment;

FIG. 5 is a detailed diagrammatic illustration of an energy generator and cooling system located on a robotic limb in accordance with an embodiment;

FIG. 6 is a detailed diagrammatic illustration of an energy generator and cooling system located on a rotating limb in accordance with an embodiment; and

FIG. 7 is a variant of detailed diagrammatic illustration of an energy generator and cooling system located on a robotic limb with connection to a rotating end effector in accordance with an embodiment.

### DESCRIPTION OF THE INVENTION

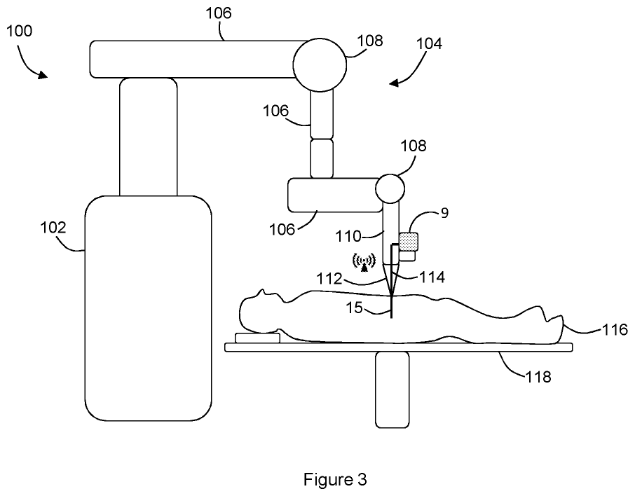

An embodiment is shown in FIG. 3 which describes a compact electromagnetic energy source 9 located on or near to the end effector limb of a surgical robot 100.

The surgical robot 100 comprises a main body 102 and a moveable robotic arm 104. The robotic arm 104 may also be referred to as a robotic actuator or manipulator. The robotic arm 104 comprises a plurality of links 106 which are coupled by joints 108. Each joint 108 provides relative motion of two links coupled by the joint, for example rotational motion and/or linear motion. At the end of the robotic arm 104 is an end effector 112.

The link adjacent to the end effector 112 may be referred to as the terminal limb 110. In the present embodiment, the end effector 112 is coupled to the terminal limb 110 by a rotatable joint. In other embodiments, the end effector 112 may be coupled to the terminal limb 110 by a fixed coupling that does not rotate. In further embodiments, the end effector 112 is incorporated into the terminal limb 110.

A radiating applicator 15 is mounted to the end effector 110. In the present embodiment, the radiating applicator 15 is a single-use, disposable device. The radiating applicator comprises a monopole antenna that is configured to radiate microwave energy into tissue. In other embodiments, the radiating applicator 15 may comprise any suitable antenna that is configured to radiate RF or microwave energy into tissue. The radiating applicator 15 may also be referred to as an energy delivery probe.

In the present embodiment, energy source 9 is mounted on the terminal limb 110 near the end effector 112. In other embodiments, the energy source 9 may be positioned in any suitable location on or near the end effector 112, for example on any link 106 of the robotic arm 104 that is near to the distal end of the robotic arm 104.

In some embodiments, the energy source 9 is configured to interface with existing mechanical and/or electrical connections on the surgical robot 100, for example connections that are designed to interface with a camera or endoscope. A form factor of the energy source 9 may be designed to fit to an existing connection or housing on the surgical robot 100.

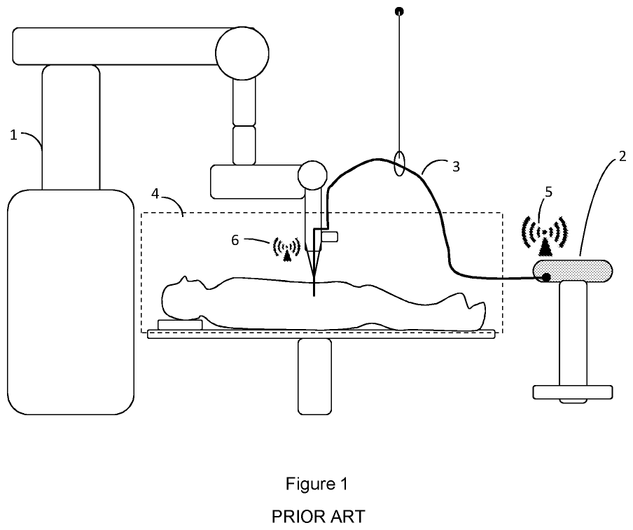

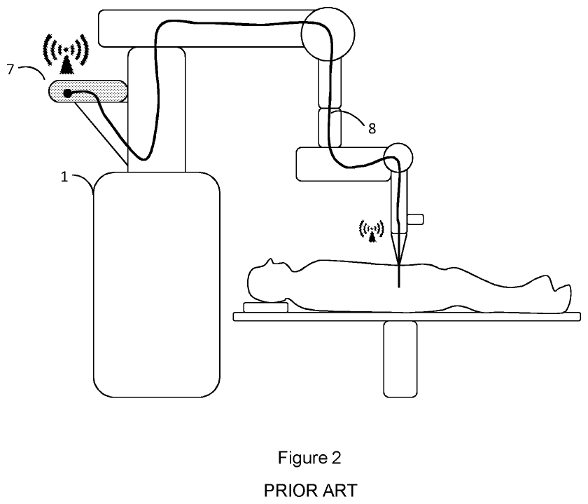

The energy source 9 comprises an electromagnetic generator. The energy source 9 is configured to supply microwave energy to the radiating applicator 15 through a coaxial cable 114. The length of the coaxial cable 114 may be much shorter than the coaxial cables 3, 8 shown in the examples of FIGS. 1 and 2. For example, a length of the coaxial cable may be 2 metres or under. In other embodiments, the length of the coaxial cable may be 1 metre or under, or 0.5 metres or under. In further embodiments, the electromagnetic generator may join directly to the antenna probe.

In this location on the terminal limb 110 as shown in FIG. 3, the electromagnetic energy source 9 can be designed to produce a much reduced level of energy in order to deliver an energy level 116 that equates with the energy previously delivered 6 in the system of FIG. 2.

The system of FIG. 3 is configured to apply radiation to a patient 116 or other subject, who in FIG. 3 is depicted as lying on a bed 118.

In use, the robot 100 positions the end effector 112 such that the radiating applicator 15 is positioned in a desired operational position relative to the patient. The desired operational position is a three dimensional physiological location within the patient 116.

In the present embodiment, a planning system (not shown) stores volume data that is representative of a volume to be treated. For example, the volume data may comprise a tumour volume that has been identified within the patient 116. The robot 100 identifies the desired operational position using the volume data, and positions the end effector 112 such that the radiation applicator is positioned to deliver microwave energy to the tumour volume.

The radiating applicator 15 may be positioned interstitially or via a catheter or by any other suitable method.

In the present embodiment, a position detection system (not shown) of the surgical robot 100 is used to determine the position of the radiating applicator 15, for example a position of the radiating applicator 15 relative to the desired three dimensional physiological location. The position detection system may be used via computer numerical control to locate the effector and probe in x,y,z space, Cartesian space or in any relative coordinate system. In other embodiments, any method of position detection may be used.

The energy source 9 generates microwave energy which is supplied to the radiating applicator 15 via the coaxial cable 114. In the present embodiment, the microwave energy has a frequency between 900 MHz and 30 GHz, for example 915 MHz or 2.54 GHz. In other embodiments, the energy source may generate energy at any suitable RF or microwave frequency, for example any suitable frequency between 1 KHz and 300 GHz. The microwave energy supplied may be amplitude-modulated or pulse width-modulated.

The radiating applicator 15 radiates microwave energy into the tissue of the patient 116. The microwave energy heats the tissue into which it is radiated, and may thereby perform ablation or tissue heating of a target region of tissue.

The embodiment of FIG. 3 may be considered to provide an improvement relative to the examples illustrated in FIG. 1 and FIG. 2. The method of FIG. 3 may provide an improved method of use of an energy generator via robotic placement. Energy losses may be reduced. A risk of the cabling tangling or breaking may be reduced. There may be less risk of crushing or kinking of the cable, which may lead to reflection or absorption of energy. The cable arrangement may not limit the robot's freedom of movement. Less cooling may be used when compared to the examples of FIG. 1 and FIG. 2, which may also reduce the system's complexity and/or expense.

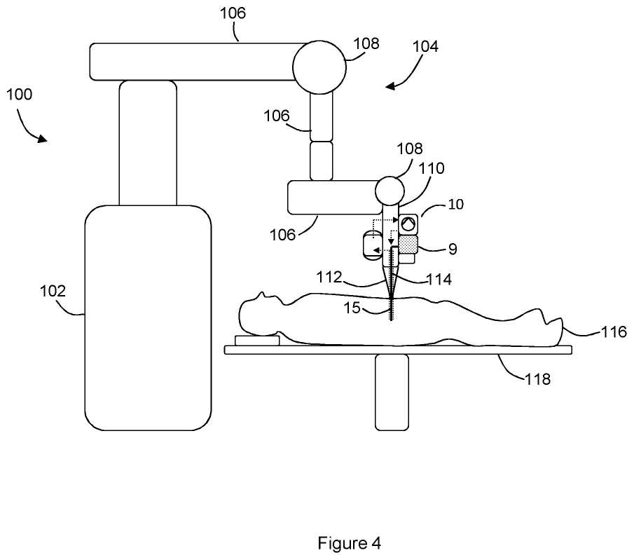

In a further embodiment illustrated in FIG. 4 a cooling system 10 is incorporated into a system similar to that of FIG. 3. In the embodiment of FIG. 4, the cooling system 10 is positioned adjacent to the electromagnetic source 9, mounted on the terminal limb 110 near the end effector 112. In other embodiments, the cooling system 10 may be positioned in any suitable location on or near to the end effector 112.

In general, cooling systems may have issues with cabling where a cooled fluid or gas is supplied to the applicator so it may be advantageous to minimise these cable runs to reduce complexity, avoid damage and to promote a more compact tool manageable solution for the robot. In some embodiments, the cooling cabling, medium (e.g. fluid water/saline or gas CO2 or N) and applicator 15 form a single replaceable item.

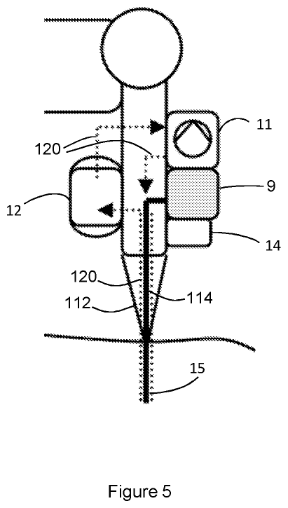

A more detailed description of the arrangement of FIG. 4 is presented in FIG. 5. The cooling system 10 comprises a pump 11. The cooling system 10 further comprises a reservoir 12, which may also be referred to as a tank or coolant container. The cooling system 10 further comprises cooling cabling which transfers coolant between the pump 11 and reservoir 12, and around the radiating applicator 15 to provide cooling of the radiating applicator 15. A path taken by coolant through cooling cabling is shown by dotted lines 120.

The energy system 9 is seated on a robotic limb 110 on a pedestal or interfacing plate 14. The treatment applicator 15 is cooled via the pump 11 which supplies a cooling medium taken from a reservoir or tank 12 to the applicator 15, thereby cooling a treatment site. The cooling medium is then returned to the coolant container 12 in a closed loop cycle shown by dotted lines 120. The interfacing plate 14 in addition to providing mechanical connection may be designed to be a thermal conduit for the energy system 9 to assist with sinking of excess heat created as a result of inefficiency in the energy generation circuitry. In further embodiments, a further thermal conduit may be used to sink heat from the energy source 9 and/or radiating applicator 15.

Providing a cooling system 10 that is located on a distal portion of the robotic arm 104 near to the radiating applicator 15 may reduce complexity and/or provide a more efficient cooling method.

In a further embodiment illustrated in FIG. 6 the robotic surgical system 100 is configured to provide rotational control 17 of a directional antenna 19 where the energy source 9 is located close to the treatment site. The directional antenna 19 has a radiation pattern that depends on an angle around the longitudinal axis of the directional antenna 19. Therefore, rotating the directional antenna 19 may allow higher (or lower) amounts of energy to be radiated into certain areas of tissue.

In this configuration the limb 110 that contains the energy source 9 (and alternatively or additionally the cooling system 10) may be entirely rotated. This means that the terminal limb 110 may have a fixed direction 16 that the end effector limb 112 initially shares.

A rotational angle of the terminal limb 110 is represented by arrow 16 in FIG. 6. A rotational angle of the end effector 112 is shown by an arrow 18 and a rotational angle of the directional antenna 19 is shown by an arrow 20. In FIG. 6, the terminal limb 110, end effector 112 and directional antenna 19 have a common angle and may be rotated together.

In some embodiments, the end effector 112 may have a rotational freedom that permits axial rotation to a new location 20 that is also shared with the directional antenna 18.

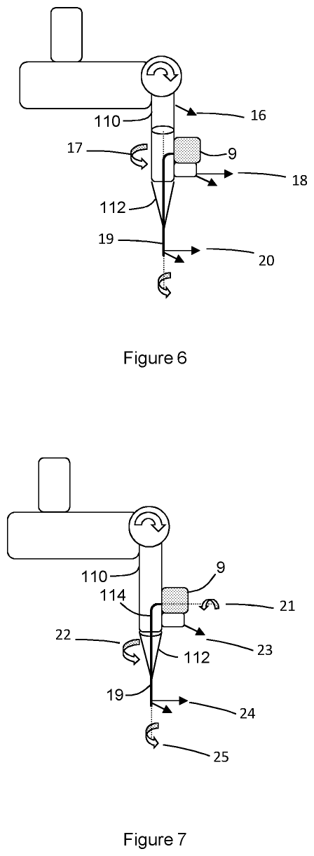

In an embodiment depicted in FIG. 7 only the end effector 22 rotates to change the axial position 25 of a directional antenna 19 to a new location 24 different to the starting location 23. This places a torque 21 on the applicator cabling which can be accommodated by using a rotating coaxial coupling (not shown) between the applicator cabling 115 and the energy source 9. Examples of such rotating connector families include, for example, SNP, SMA, BMA, N-type.

In embodiments described above, the energy source 9 is an energy generator configured to generate microwave energy from electrical energy. In other embodiments, the energy source 9 may comprise an amplifier which is configured to convert a low-energy microwave signal into a higher-energy microwave signal. A microwave generator positioned further from the end effector (for example, on the main body of the robot 100 or external to the robot 100) may produce the low-energy signal. By passing a low-energy signal through a longer cable and then passing a higher-energy signal only through a shorter length of cable from the amplifier, cable losses may be reduced.

In embodiments described above, the location of an energy source is described to improve efficiency in robotic applications. The method includes a robotic surgical device or system, electromagnetic energy generator/amplifier system, cabling and applicator used to deliver energy from a generator/amplifier system to a recipient device, for example a radiating applicator or antenna that transfers the energy into biological tissue for treatment purposes.

Energy is delivered to a radiating element from a compact electromagnetic energy source/amplifier mounted close to the end of a robotic actuator/manipulator. The purpose is to generate and deliver RF/microwave energy (1 KHz to 300 GHz) in close proximity to the point of treatment to minimise energy loss.

It may be understood that the present invention has been described above purely by way of example, and that modifications of detail can be made within the scope of the invention.

Each feature disclosed in the description and (where appropriate) the claims and drawings may be provided independently or in any appropriate combination.

1. A system comprising:
a surgical robot comprising a moveable robotic arm;
a radiating applicator positioned at a distal end of the robotic arm, wherein the robotic arm is configured to move the radiating applicator to a desired operational position; and
an energy source positioned on a distal portion of the robotic arm, in proximity to the radiating applicator,
wherein the energy source is configured to provide RF or microwave energy to the radiating applicator for radiation by the radiating applicator.

2. The system according to claim 1, further comprising a coaxial cable connecting the energy source to the radiating applicator, wherein a length of the coaxial cable is less than 2 metres.

3. The system according to claim 1, wherein the distal portion of the robotic arm comprises an end-effector of the robot arm, and wherein the radiating applicator and energy source are positioned on the end-effector.

4. The system according to claim 1, wherein the distal portion of the robotic arm comprises an end-effector of the robot arm and a further link of the robotic arm, wherein the radiating applicator is positioned on the end-effector and the energy source is positioned on the further link.

5. The system according to claim 4, wherein the distal portion of the robotic arm is axially rotatable with respect to a preceding portion of the robotic arm, and the end-effector is axially rotatable with respect to the further link of the robotic arm, so as to provide rotation of the radiating applicator independently of rotation of the energy source.

6. The system according to claim 2, wherein the distal portion of the robotic arm is axially rotatable with respect to a preceding portion of the robotic arm, and the end-effector is axially rotatable with respect to the further link of the robotic arm, so as to provide rotation of the radiating applicator independently of rotation of the energy source, and the system further comprises a rotatable coaxial coupling between the coaxial cable and the energy source and/or a rotatable coaxial coupling between the coaxial cable and the radiating applicator.

7. The system according to claim 1, wherein at least one of a) or b):
a) the radiating applicator comprises a directional antenna, and wherein robotic arm is configured to rotate the radiating applicator to provide a desired direction of radiation from the directional antenna; or
b) the system further comprises a thermal interface between the energy source and robotic arm, wherein the thermal interface is configured to pass heat from the energy source into the robotic arm for the purpose of heat sinking, wherein the thermal interface comprises a high thermal conductivity material.

8. (canceled)

9. The system according to claim 1, wherein at least one of a), b) or c):
a) the energy source comprises an energy generator configured to receive electrical energy and to generate RF or microwave energy;
b) the energy source comprises an amplifier configured to receive lower-power RF or microwave energy and to generate higher-power RF or microwave energy; or
c) the robotic arm comprises a power connection for powering peripheral devices and/or tools, and the energy source is configured to connect to the power connection.

10. (canceled)

11. (canceled)

12. The system according to claim 1, wherein the system further comprises a controller configured to control at least one parameter of the energy provided by the energy source.

13. The system according to claim 12, further comprising a communications device configured to obtain data relating to the energy provided by the energy source and/or an effect of the energy provided by the energy source, and to send the data to the controller;
wherein the controller is configured to control the at least one parameter of the energy provided by the energy source based on the data sent by the communications device.

14. The system according to claim 13, wherein at least one of a) or b):
a) the communications device comprises or is coupled to at least one sensor; or
b) the data comprises at least one of a system temperature, an applicator temperature, forward power, reflected power, duty cycle, antenna direction, antenna rotational speed, advancement rate, or withdrawal rate.

15. (canceled)

16. The system according to claim 12, further comprising a position detector system configured to output position data that is representative of a position of the radiating applicator, wherein the controller is configured to control the energy source in dependence on the position data.

17. The system according to claim 16, wherein controlling the energy source in dependence on the position data comprises at least one of a) or b):
a) controlling energy during applicator withdrawal to perform surgery tract ablation; or
b) controlling energy delivered by the energy source based on volume data held in a planning system, the volume data comprising the desired operational position.

18. The system according to claim 1, wherein at least one of a) or b):
a) the robotic arm comprises a mechanical mounting adapter configured for mounting at least one peripheral device and/or tool, and the energy source is mounted on the mechanical mounting adapter; or
b) the system further comprises a cooling system positioned on the distal portion of the robotic arm, in proximity to the radiating applicator, wherein the cooling system is configured to cool the radiating applicator by circulation of a coolant fluid through at least one coolant channel.

19. (canceled)

20. A system comprising:
a surgical robot comprising a moveable robotic arm;
a radiating applicator positioned at a distal end of the robotic arm, wherein the robotic arm is configured to move the radiating applicator to a desired operational position; and
a cooling system positioned on a distal portion of the robotic arm, in proximity to the radiating applicator,
wherein the cooling system is configured to cool the radiating applicator by circulation of a coolant fluid through at least one coolant channel.

21. The system according to claim 20, wherein a length of the coolant channel is less than 3 metres.

22. The system according to claim 20 wherein the distal portion of the robotic arm comprises an end-effector of the robot arm, and wherein the radiating applicator is positioned on the end-effector; and
the distal portion of the robotic arm further comprises a further link of the robotic arm, wherein the radiating applicator is positioned on the end-effector and at least part of the cooling system is positioned on the further link,
the end-effector is axially rotatable with respect to the further link of the robotic arm, so as to provide rotation of the radiating applicator independently of rotation of the at least part of the cooling system; and
the system comprises a rotatable coupling configured to connect the at least one coolant channel to the at least part of the cooling system.

23. The system according to claim 22, wherein the at least part of the cooling system comprising a pump and a coolant reservoir.

24. A method comprising:
moving, by a robotic arm of a surgical robot, a radiating applicator to a desired operational position, wherein the radiating applicator is positioned at a distal end of the robotic arm; and
providing, by an energy source, RF or microwave energy to the radiating applicator for radiation by the radiating applicator, wherein the energy source is positioned on a distal portion of the robotic arm, in proximity to the radiating applicator.

25. A method comprising:
moving, by a robotic arm of a surgical robot, a radiating applicator to a desired operational position, wherein the radiating applicator is positioned at a distal end of the robotic arm; and
cooling, by a cooling system, the radiating applicator by circulation of a coolant fluid through at least one coolant channel, wherein the cooling system is positioned on a distal portion of the robotic arm, in proximity to the radiating applicator.
